(19) United States
(12) Patent Application Publication
(10) Pub. No.: US 2010/0105129 A1
(43) Pub. Date: Apr. 29, 2010

(54) Biomass production system

(76) Inventors: Jose L. Sanchez-Pina, Los Mochis (MX); Ramon Sanchez-Pina, Los Mochis (MX)

Correspondence Address: LAMBERT & ASSOCIATES, P.L.L.C., 92 STATE STREET, BOSTON, MA, 02109-2004, US

(21) Appl. No.: 12/290,044

(22) Filed: Oct. 27, 2008

(51) Int. Cl. C12M 1/09; C12M 3/00

(52) U.S. Cl. 435/286.5; 435/292.1

(57) ABSTRACT

A system preferably for light irradiation, wherein the system includes a support structure, wherein the support structure is able to accommodate an array of vessels capable of receiving light; the system includes a buoyancy support system and a feeding harvesting system, wherein the buoyancy support system is in fluid and data communication with the array of vessels to regulate the amount of light introduced into the vessels within a fluid medium.

### BACKGROUND OF THE INVENTION

1. Field of the Invention

The present invention relates generally to energy production, and more particularly, to the production of micro algae biomass.

2. Description of the Related Art

With the increase in demand of energy sources, biofuel has emerged as an alternative to fossil fuel. Today biofuel is obtained from biomass, specifically terrestrial plants producing organic matter or combustible oil. Crops such as soybeans, canola, palm oil, corn, and sugarcane are commonly used to produce biodiesel, ethanol, biogas, and renewable hydrogen. However, harvesting terrestrial plants to produce biofuel is often inefficient due to low yields per hectare and the need for a large amount of land and water required for proper growth. Consequently, a great strain is placed on environmental resources, inducing competition between the biofuel and food industries, resulting in a decreased quantity of biofuel at a greater cost of production. Thus, with current methods, fuels obtained from terrestrial plants may be unable to compete with fossil fuels in meeting short term energy requirements.

As a better biofuel-producing candidate, micro algae have shown to be efficient converters of solar energy into biomass due to their simple cellular structure and ability to grow in aqueous suspension where access to water, carbon dioxide, and simple nutrients is plentiful. Given their simple characteristics, micro algae are theoretically capable of producing several times the amount of useful biomass per unit area of land, if compared to terrestrial counterparts. Furthermore, these organisms do not require the use of freshwater, large tracts of land suitable for agriculture and/or complex fertilizers, and thus, would not compete with conventional food producing crops for limited environmental resources.

Several systems which employ micro algae are in use today. One such system is named “ponds in air” and involves digging a continuous trench in the soil filled with water and inoculated with a micro algae culture. The system also includes a paddle wheel to generate a continuous flow of water to mix nutrients and carbon dioxide. Another system, known as “photo bioreactor indoors” utilizes a transparent vessel that is filled with water and inoculated with a micro algae culture. Unlike “ponds in air,” which is usually used in “open air,” this system is usually indoors so that the amount of light, carbon dioxide, fertilizer, microorganisms, and temperature may be controlled to allow for optimum cell growth. Lastly, another system, known as “photo bioreactor outdoors,” is similar to “photo bioreactor indoors” except, as the name implies, the system exists outdoors, whereby carbon dioxide is generally provided by the effluent of a thermal power generating facility.

While each abovementioned system may be preferable to terrestrial plant systems, there are nevertheless some disadvantages pertaining to each respective system. One major disadvantage of the “open pond” system involves uncontrolled environmental fluctuations due to the system being open to the elements. Common problems include inconsistent carbon dioxide concentrations, contamination from other algal species and micro organisms, changes in water temperature, and susceptibility to light saturation. These problems can be avoided in “indoor photo bioreactors,” wherein environmental conditions can be controlled. However, the cost of controlling growth conditions is very expensive and the size of the system is greatly limited as a result of being indoors. Similarly, “outdoor photo bioreactors” may control certain environmental conditions but invariably fall prey to the same deficiencies found in “open pond” systems.

In light of this, the present invention relates to a system that combines reliable and inexpensive technologies to emulate conditions at sea that are optimum for growth of micro algae biomass.

### SUMMARY OF THE INVENTION

The instant invention, as illustrated herein, is clearly not anticipated, rendered obvious, or even present in any of the prior art mechanisms, either alone or in any combination thereof.

The primary object of the instant invention is to provide a way to perform off-shore micro-algae farming in order to harvest the solar irradiation of large areas-of the world to fulfill the increasing energy demands of emerging economies and developed countries without a significant modification of the environment.

Another object of the instant invention is to provide an inexpensive way to control the water temperature and light irradiation that enclosed photo-bioreactors receive in order improve the production yield of biomass and have a better quality of carbohydrates and fats to produce bio-fuels.

Another object of the instant invention is to provide a way to produce biomass for bio-fuels that minimizes the use of freshwater and farmland, without the use of genetic modification of edible grains or feedstock in order to decrease the likelihood affecting the international prices of food for human and animal consumption.

Another object of the instant invention is to provide a way to control the amount of nutrients, catalysts and carbon dioxide that is introduced into a biomass production system in order to increase the process yield and provide certainty about the cost effectiveness of the operation (because the influence of the weather is minimized).

Another object of the instant invention is to provide a process that regulates the amount of carbon dioxide reuse or true carbon sequestration to reduce the likelihood of climate change effects by reducing the rate of Green-House-Gases emissions to the atmosphere.

Another object of the instant invention is to provide a way to do micro-algae farming that reduces the likelihood of nutrients and/or algae strain leaks to the ecosystem (that could potentially generate algae blooms and low oxygen areas in the ocean with no fish or other maritime resources).

Another object of the instant invention is to reduce the prices of bio-fuels in order to make them a competitive alternative to oil products, which in turn might help to improve air quality in different areas around the world.

Another object of the instant invention is to provide a way to perform micro-algae. farming that does not require the destruction of habitats such as coastal areas, estuaries or flat land in proximity to a body of water.

Another object of the instant invention is to provide a way to produce bio-fuels for nations and islands with access to a sea or large body of water and very little flat or arable land; this could potentially help these nations to achieve energy independence.

Another object of the instant invention is to provide a way to protect the infrastructure of a renewable energy operation (micro-algae farm) in case of severe weather conditions such as storms and hurricanes.

Another object of the instant invention is to create re-fuel stations in the middle of the ocean to increase the value-added area for cargo in current boats, reduce the size of new cargo ships so they can operate in shallow harbors (more access of merchandise to people in underdeveloped countries) thus facilitating maritime transportation and global commerce.

Another object of the instant invention is to avoid the construction of large artificial islands that could become breeding grounds for birds and sea mammals, thus affecting the maritime ecosystem.

Another object of the instant invention is to provide new jobs for fishermen and other people that have been affected by the depletion of fish species in different coastal areas around the world due to over fishing and low oxygen marine environments due to an excess of nutrients from agricultural and industrial run-off.

There has thus been outlined, rather broadly, the more important features of the biomass production system in order that the detailed description thereof that follows may be better understood, and in order that the present contribution to the art may be better appreciated. There are additional features of the invention that will be described hereinafter and which will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

These together with other objects of the invention, along with the various features of novelty, which characterize the invention, are pointed out with particularity in the claims annexed to and forming a part of this disclosure. For a better understanding of the invention, its operating advantages and the specific objects attained by its uses, reference should be made to the accompanying drawings and descriptive matter in which there are illustrated preferred embodiments of the invention.

### BRIEF DESCRIPTION OF THE DRAWINGS

FIG. 1A illustrates a biomass production system, having a support structure that is able to accommodate an array of vessels, and wherein the system includes a buoyancy support system and harvesting feeding system connected to the vessels.

FIG. 1B illustrates an example of mid solar irradiation levels penetrating the array of vessels based upon the location of the sun.

FIG. 1C illustrates an example of high solar irradiation levels penetrating the array of vessels based upon the location of the sun.

FIG. 1D illustrates an example of low solar irradiation levels penetrating the array of vessels based upon the location of the sun.

FIG. 2 illustrates the support structure and vessels of the system for biomass production, wherein micro algae treated with nutrients and metabolic catalysts are placed for growth and production of biomass.

FIG. 3 illustrates a light sensing device, which is placed within the array of vessels to measure the amount of light penetrating the vessels throughout the day.

FIG. 4 illustrates a depth control processor unit that is in data and electrical communication with the light sensing device to determine whether the depth of the array of vessels needs to be adjusted within a fluid medium depending upon the amount of light irradiation.

FIG. 5 illustrates an operational system flowchart for data communication between the light sensing device and the depth control processing unit via a wireless transmission.

FIG. 6 illustrates the operational system flowchart for data communication between the depth control processor unit and a solenoid valve of the buoyancy support system to introduce compressed air into the system to raise or lower the vessels.

FIG. 7 illustrates the harvesting and feeding operations of the instant invention, specifically how biomass is extracted from the vessels along with treating the micro algae with various nutrients and metabolic catalysts.

FIG. 8 illustrates the connection between the harvesting and feeding operations of the instant invention and the array of vessels.

FIG. 9 illustrates an alternate embodiment of the biomass production system, wherein the system includes identical buoyancy support systems connected to the array of vessels.

### DETAILED DESCRIPTION OF THE PREFERRED EMBODIMENT

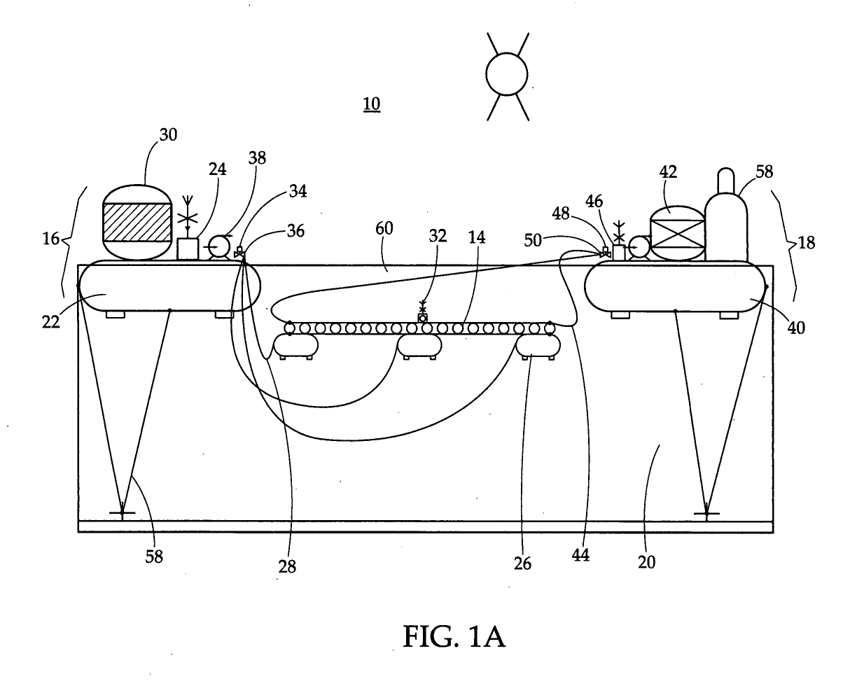

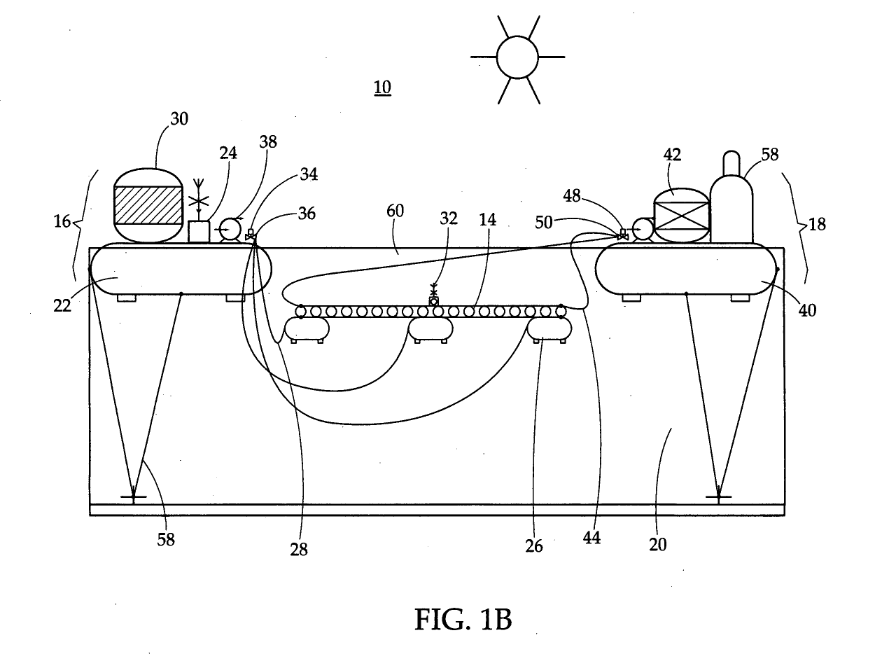

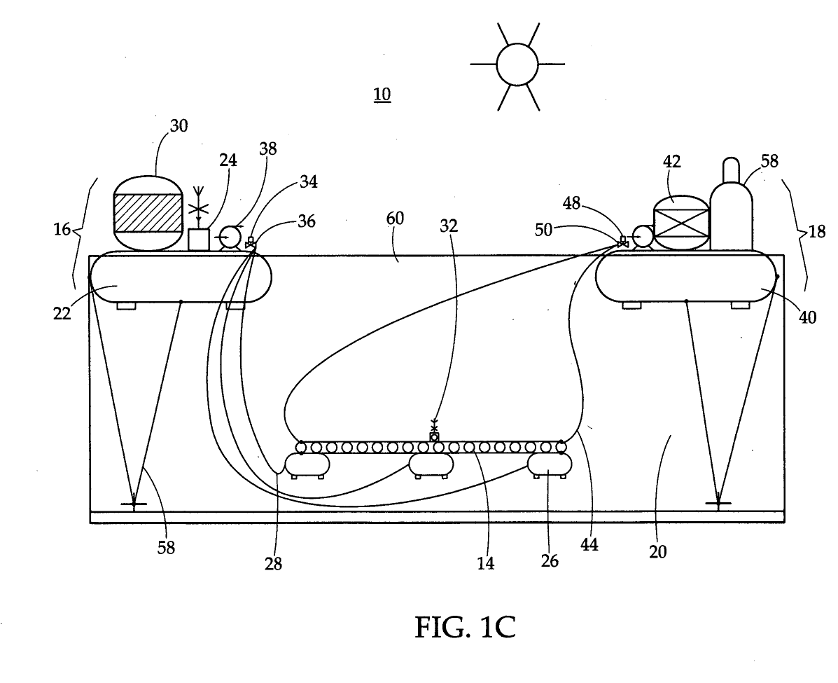

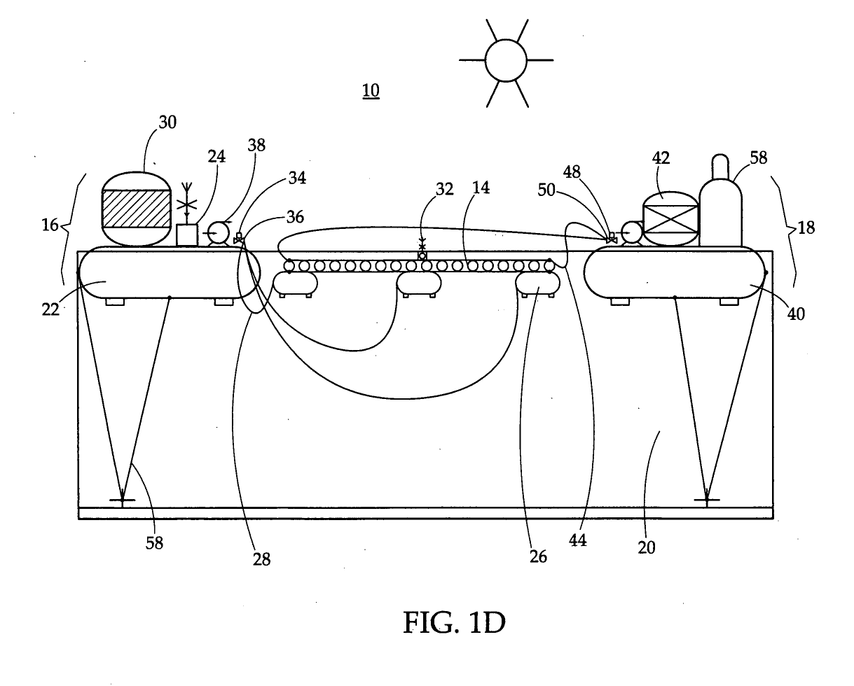

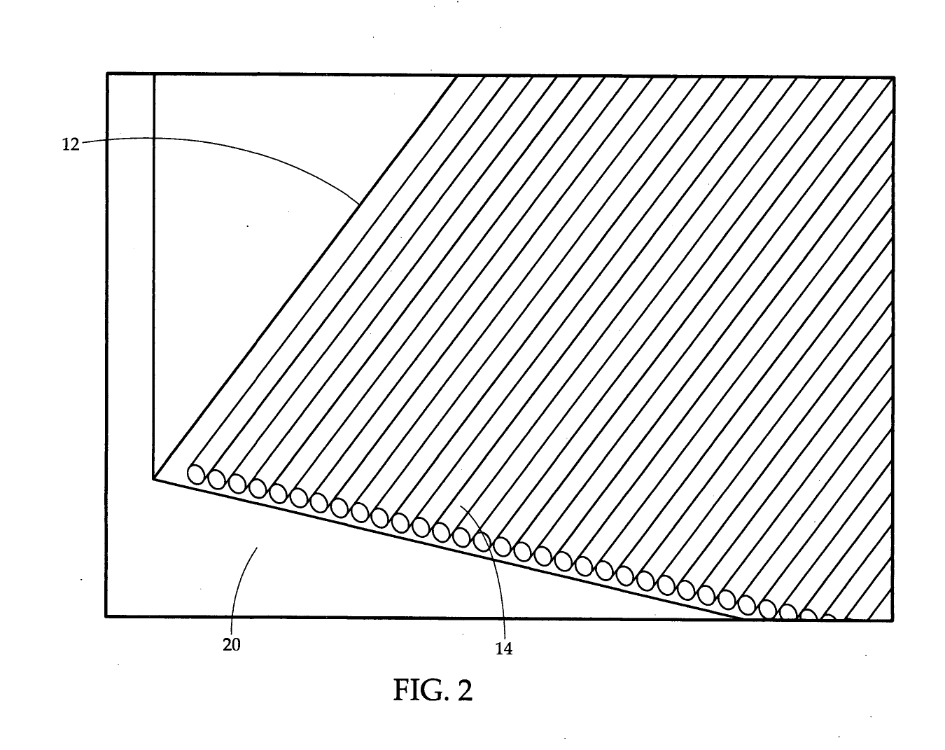

FIG. 1A illustrates a system 10 preferably for light irradiation, wherein the system 10 includes a support structure 12 (see FIG. 2), wherein the support structure 12 is able to accommodate an array of vessels 14 capable of receiving light. The system 10 includes a buoyancy support system 16 and a feeding harvesting system 18, wherein the buoyancy support system 16 is in fluid and data communication with the array of vessels 14 to regulate the amount of light introduced into the vessels 14 within a fluid medium 20. Preferably the fluid medium 20 is a body of water, and in yet a more preferable embodiment, the system 10 is operable in an open body of water such as the ocean.

The buoyancy support system 16 further includes a floating support 22, wherein the floating support 22 preferably resides on the surface of the fluid medium 20 to provide stability to the system 10 and allow for the array of vessels 14 to remain substantially constant in position. Furthermore, the floating support 22 includes a depth control processing unit 24 that works in conjunction with a plurality of depth controllers 26 via flexible compressed air piping 28 to regulate the depth of the array of vessels 14 within the fluid medium 20. Additionally, the floating support 20 includes a compressed air storage tank 30, wherein the tank 30 allows for the introduction of compressed air into each depth controller 26 such that altering the amount of compressed air within the depth controller 26 causes the array of vessels 14 to receive varying amounts of light.

The depth control processing unit 24 regulates the amount of compressed air introduced into each depth controller 26 based on a signal sent from a light-sensing device 32 (see FIG. 2) located within the array of vessels 14. The light sensing device 32 measures the amount of light received by the array 14 and subsequently transmits the signal to the depth control processing unit 24 such that the processing unit 24 determines whether the amount of light received by the array 14 is within normal operating parameters, or alternatively whether the distance between the vessels 14 and surface of the fluid medium 20 must be adjusted. The floating support 20 further includes a solenoid valve 34, wherein the solenoid valve 34 regulates an air valve 36 to maintain proper air flow into and out of the depth controllers 26 from the air storage tank 30. A pump 38 is utilized within the floating support 20 to transfer compressed air to the depth controllers 26 based on the signal sent from the light sensing device 32 to the depth control processing unit 24. As such, the depth control processing unit 24 is in electrical communication with the pump 38 and solenoid valve 34 to either activate or deactivate the air valve 36 based on the light reading from the light-sensing device 32. As stated above, the depth of the vessels 14 is adjusted by the system 10 introducing compressed air into the depth controllers 26 via the piping 28 and allowing the vessels 14 to raise or lower until the proper light setting is reached.

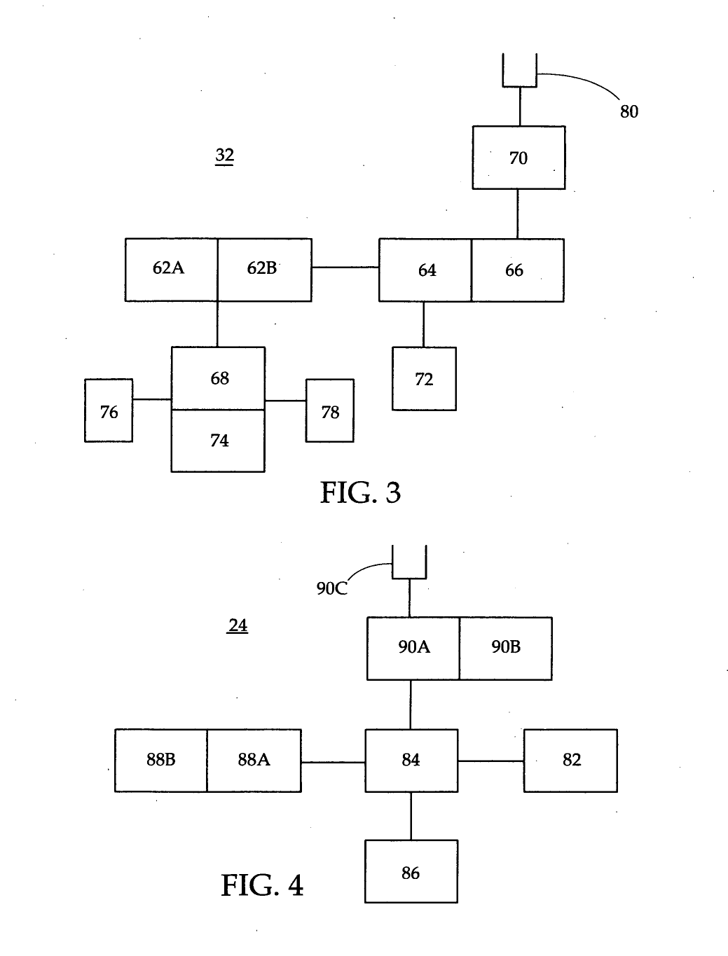

In the preferred embodiment, the system 10 is configured for the production of biomass, preferably using micro algae in conjunction with oceanic conditions which are optimal for the growth of micro algae biomass. As described above, the system is preferably operable in the ocean, given the near constant water temperature and the vast area in which to deploy the system 10. Therefore, in operation, each vessel 14 includes a predetermined amount of micro algae utilized for the production of biomass through the introduction of various nutrients along with varying degrees of exposure to light, preferably sunlight. Once the micro algae is introduced into the vessels 14, the vessels 14 are submerged in the water at a depth that provides the amount of sunlight necessary for optimal growth of the micro algae. Furthermore, as described above and shown in FIG. 3, the light-sensing device 32 located on the array of vessels 14 constantly measures the amount of sunlight penetrating each vessel 14 and relays the measurement to the depth control processing unit 32 to determine whether the depth of the vessels must be adjusted. As known in the art, over the course of a day, the position of the sun changes thereby affecting the amount of sunlight reaching each vessel 14 at a specific depth, therefore it is preferable to raise and lower the vessels 14 to maintain a constant amount of light penetrating the vessels 14 thus ensuring optimal growth conditions throughout the course of a day (see FIGS. 1B, 1C & 1D).

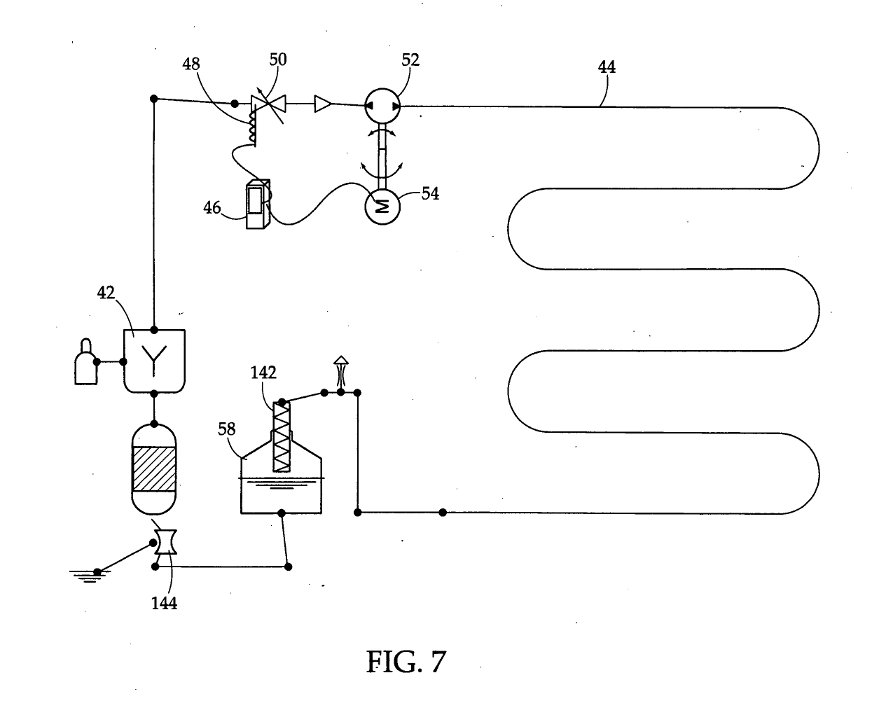

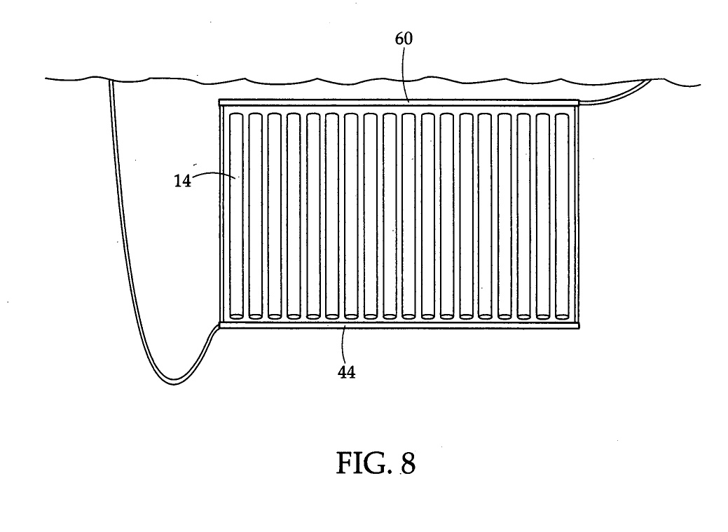

The feeding harvesting system 18 possesses dual functions, namely the system 18 is responsible for the introduction of various nutrients, carbon dioxide and metabolic catalysts to the micro algae located within the vessels 14 for optimal growth. The feeding harvesting system 18 also regulates the harvesting and gathering of the biomass produced by the micro algae within the vessels 14 for storage and eventual transport for use in a variety of applications. In the preferred embodiment, the feeding system 18 further includes a floating support 40, wherein the floating support 40 comprises a storage tank 42 for a mixture of carbon dioxide, nutrients and metabolic catalysts that will be introduced into the vessels 14 containing the micro algae via a nutrient piping 44. The feeding system 18 also includes a biomass storage tank 58 for all harvested micro algae biomass contained within the array of vessels 14. The feeding and harvesting system 18 regulates the introduction of nutrients and the removal of biomass from the system 10 via a feed/harvest control unit 46 that activates/deactivates a solenoid valve 48 which controls an air valve 50. Furthermore, the feed/harvest control unit 46 is in electrical communication with the solenoid valve 48 and a pump 52 that activates a motor 54 to introduce treated water containing nutrients and metabolic catalysts into the vessels 14 for biomass production (see discussion below and FIGS. 7 & 8). The feed/harvest control unit 46 also regulates the harvesting and removal of biomass from the vessels 14 for placement in the biomass storage tank 58 via a harvesting piping 60. The feed/harvest control unit 46 is also in data and electrical communication with the depth control processor unit 24 to regulate the timing cycle for both introducing the nutrients into the vessels 14 and removing the biomass.

In one embodiment of the instant invention, the system 10 may include a plurality of anchors 56, wherein the anchors 56 secure the system to the bottom of the fluid medium 20, such as a seabed, by connection to the buoyancy floating support 22 and the harvesting feeding floating support 40.

FIGS. 1B, 1C and 1D illustrate the varying solar irradiation levels throughout a given day, wherein the location of the sun in relation to the system 10 varies, thereby causing the system 10 to raise or lower to acclimate to the normal operating limits. FIG. 1B illustrates a level of mid-solar irradiation, while FIG. 1C illustrates a level of high solar irradiation and FIG. 1D illustrates a level of low solar irradiation. As can be seen from the various figures, depending on the amount of light penetrating the array of vessels 14, the depth of the vessels 14 in relation to the surface of the fluid medium 20 changes throughout the course of the day to maintain constant light irradiation, thereby creating optimal growth conditions for the micro algae. Thus, during high levels of solar irradiation, the array of vessels 14 must be lowered through the process described above to prevent too much light exposure thereby limiting biomass production, conversely, during low levels of solar irradiation, the vessels 14 must be raised close to the surface of the fluid medium 20 to ensure enough light penetrates the vessels 14 and reaches the micro algae.

It is known in the art that each species of micro algae possesses a specific tolerance to the amount of light in regards to ideal growth conditions; therefore, as discussed below and in the experimental section, the system 10 is designed to constant measure the amount of light penetrating the vessels 14, and through preset conditions within the depth control processing unit 24, possesses the ability to raise or lower the array 14 to ensure the amount of light is within normal operating parameters for optimal growth of the micro algae being used in the system 10, as discussed below.

FIG. 2 illustrates the support structure 12 and array of vessels 14 of the instant system 10, wherein the support structure 12 accommodates the array of vessels 14. In this embodiment, it is shown that the array of vessels 14 is horizontally disposed above the support structure 12. Further, in this embodiment, the support structure 12 consists of a substantially square perimeter, wherein a pair of perpendicular support members extends from near the mid-point of each side of the square perimeter to form a cross-like shape. In alternate embodiment, various other designs and shapes may be utilized to form the support structure 12 and house the array of vessels 14 depending on the amount and size of the vessels 14 along with the environment of the fluid medium 20. In one alternate embodiment, a plurality of support members is in parallel alignment with each other, and in a perpendicular alignment with the array of vessels 14.

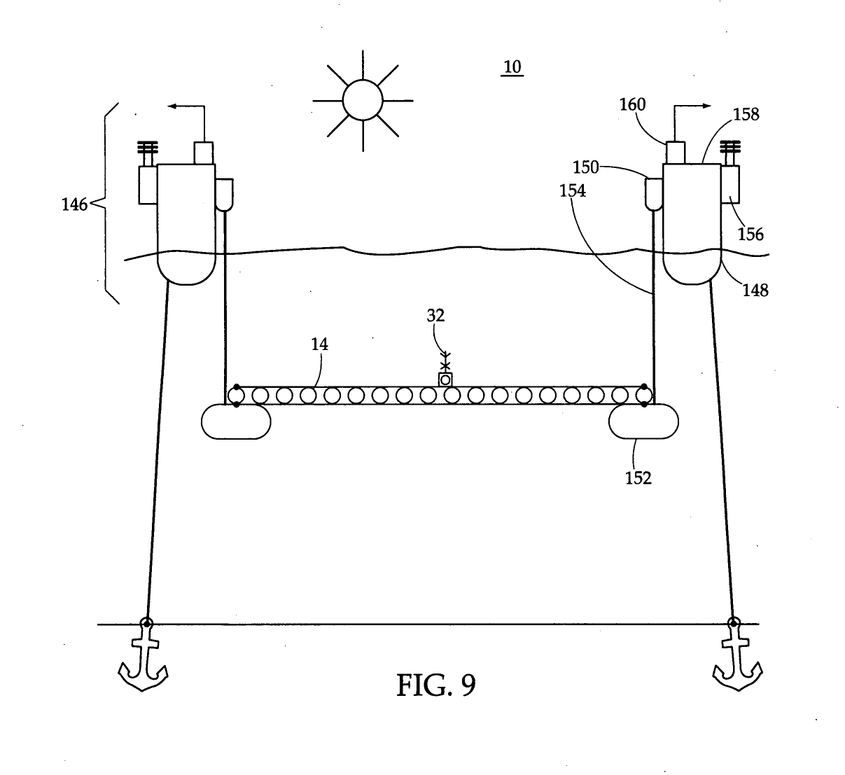

In the preferred embodiment, a cylindrical vessel is utilized, however in alternate embodiments the shape of the vessels includes, but is not limited to triangular, rectangular, semi-circular, and trapezoidal. Furthermore, as discussed below, each vessel 14 receives nutrients and other materials via the nutrient piping 44 (see FIG. 9) to assist in the growth and production of biomass. The specific design and dimensions of each vessel 14 will vary depending on the location and use of the overall system 10, and as such, it may be desirable to alter the length, circumference and thickness of the vessels 14, as well as the material used to construct each vessel 14. In one preferred embodiment, the vessels 14 are plastic, however other materials may be utilized to increase or decrease the amount of light that penetrates each vessel 14. Each vessel 14 contains a predetermined amount of micro algae that is combined with ozone pre-treatment of water prior to deployment of the system 10.

FIG. 3 illustrates the light sensing device 32, wherein the device 32 is located in the array of vessels 14. In the preferred embodiment, one device 32 is substantially centrally oriented within the array 14, however in an alternate embodiment; multiple devices 34 may be disposed equidistantly along the perimeter of the array of vessels 14. In yet another embodiment, multiple devices 32 in electrical communication and aligned with a regular system to perform iterations of testing, for correction, similar to an error function device, may be placed in various positions to compensate for the fluctuations in water temperature and sunlight irradiation, thereby ensuring proper monitoring of the overall system 10. It is known in the art, that there are no significant light irradiations or water temperature variances within a few hundred meters in any location of the array of vessels 14, or at least the variances are not significant enough to affect the production of biomass.

The light sensing device 32 includes light and temperature sensors 62A and 62B to measure the amount of light penetrating the array of vessels 14 and the water temperature in which the system is deployed. The sensors 62A and 62B are attached to an electronics circuit board 64 that has a connection to an eight-bit microcontroller 66 that is known in the art with wireless communication capabilities. The light sensing device 32 further includes a microprocessor 68 connected to the light and temperature sensors 62A and 62B, wherein the microprocessor 68 is in communication with the depth control processor unit 24 via a radio frequency (“RF”) transmitter 70, wherein the transmitter 70 preferably operates at either three hundred fifteen Mega Hertz or four hundred eighteen Mega Hertz; it is known in the art that these are the frequencies that can be used for this type of system according to FCC charts for-frequencies. The electronic circuit board 64 includes a power source 72, preferably a battery in order to power the sensors, microcontroller and RF transmitter. The micro-processor 68 further comprises a set amount of memory 74, a battery voltage monitor function 76 and an error detection function 78, wherein the error detection function 78 would send a signal to the Depth Control Processor Unit 24 to indicate that the array of vessels 14 has to go to the surface in case the power source 72 has to be replaced or there is a malfunction with either of the sensors 62A and 62B. The transmitter 70 includes an antenna 80 for wireless transmission, wherein the antenna 80 would either be imprinted into the electronics circuit board 64 or attached to the circuit board 64 and encapsulated with the entire light sensing device 32 to protect it from water intrusion.

FIG. 4 illustrates the depth control processing unit 24, wherein the processing unit 24 includes a microprocessor 82, preferably with one gigabyte of virtual memory and at least twenty gigabytes of memory in the hard drive. The processing unit 24 further contains an electronic circuit board 84, wherein the circuit board includes a power source 86, preferably a battery, thus creating quick changing capabilities in order to facilitate maintenance. The circuit board 84 is in data and electrical communication with a display 88A and keyboard 88B that facilitates programming of the system 10 to different settings, specifically involving the operating parameters for light irradiation based on the specific micro algae utilized for biomass production. In one embodiment, the circuit board 84 is enclosed within a housing to protect from water intrusion; additionally the components of the circuit board 84 are encapsulated within a resin that allows continual functioning, even if the entire system 10 is submerged within the fluid medium 20. A radio frequency transmitter (“RF”) 90A and receiver module 90B is attached to the circuit board 84 and in data communication with the light sensing device 32, to relay information about the amount of light penetrating the vessels 14 and whether it is necessary to raise or lower the array 14 based on current readings from the light sensing device 32. In a preferred embodiment, the receiver module 90B is set to an operating frequency of either three hundred fifteen or four hundred eighteen mega hertz, allowing for short range communications with the light sensing device 32. Additionally, the RF transmitter 90A contains an antenna 90C, that is capable of providing both long and short range communications, wherein the short range communications are used for the normal operation of depth control of the system 10, wherein long range communications, may include but are not limited to reporting malfunctions in the system and activating security procedures to protect the system 10 from severe weather. In one embodiment, the system 10 includes a security feature in the event of severe weather such as a hurricane to protect the overall system residing on the surface of the fluid medium 20.

In particular, the depth control processing unit 24 may be designed and programmed to receive a remote order to lower the array of vessels 14 to its deepest operating level while simultaneously beginning an emergency fast extraction of biomass from the system (see below discussion regarding normal extraction procedures), and to shut down the complete operation of the system 10 until a new order to re-start the system 10 is received by the depth control processing unit 24. Furthermore, the depth control processing unit 24 possesses an internal clock and calendar in order to be able to discriminate and reject potential mis-readings from the light sensing device 32. Lastly, the depth control processing unit 24 is in data and electrical communication with the feed/harvest control unit 46 in order to receive feedback from any potential problems that would require maintenance.

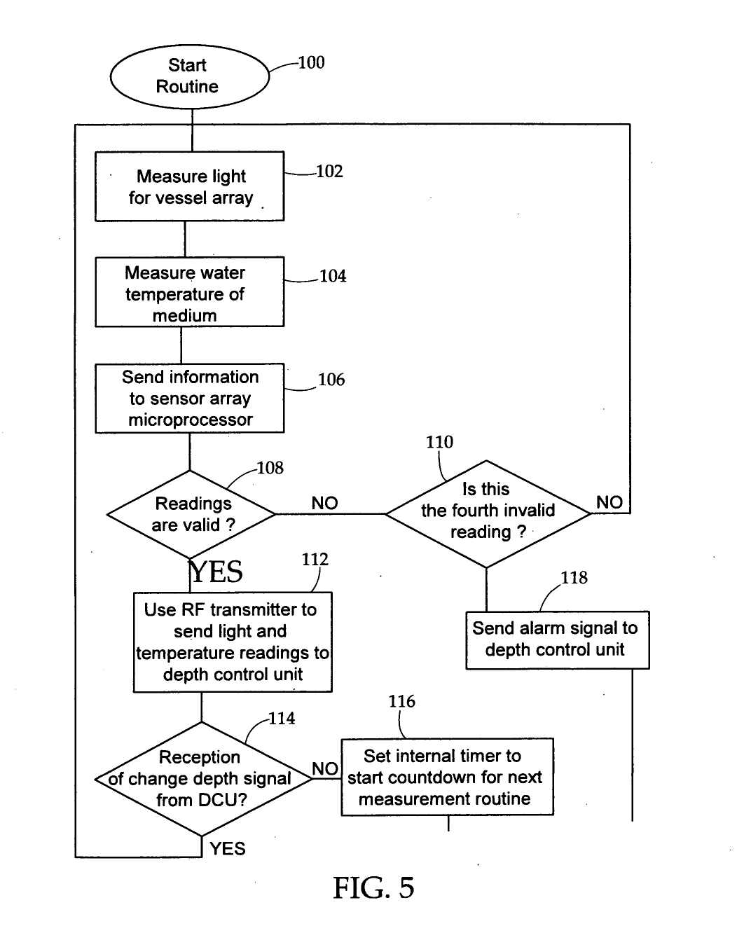

FIG. 5 illustrates an operational system flowchart for data communication between the light sensing device 32 and the depth control processing unit 24 via a wireless transmission. The purpose of the data communication is to send real-time water temperature and light irradiation conditions measured by the light sensing device 32 to the processing unit 24 to determine whether a depth modification is required to maintain optimal growth conditions for the array of vessels 14.

The data communication begins at step 100, wherein an operator of the system 10 gives a command to start the measurement cycle by the light sensing device 32. At step 102, the light sensor 62A contained within the light sensing device 32 measures the amount of light irradiation penetrating the array of vessels 14. At step 104, the temperature sensor 62B of the light irradiation device 32 measures the water temperature in which the system 10 is deployed. Once the measurements have been recorded, at step 106, the temperature and light readings are relayed to the microprocessor 68 of the light sensing device for analysis prior to transmission to the depth control processor unit 24. At step 108, the microprocessor 68 determines whether the light and temperature readings from the sensors 62A and 62B are valid in order to relay to the depth control processor unit 32.

At step 110, when the microprocessor 68 determines that the readings by the light and temperature sensors 62A and 62B are not valid, the microprocessor 68 determines whether or not this is the fourth invalid reading, and if it is not, the operation returns to the main loop at step 100 for new readings. Alternatively, if the microprocessor 68 determines the readings are valid, at step 112 the light sensing device 32 relays the recorded measurements to the depth control processor unit 24 through the RF transmitter 70 via the microprocessor 68 of the light sensing device 32. Once the recorded measurements have been sent to the depth control processor unit 24, if a depth modification is required then at step 114, the operation will return to the main loop at step 100 and the light sensing device 32 will constantly monitor the real-time light and temperature conditions until the new depth modification is achieved.

Alternatively, when no depth modification is sent by the depth control processor unit 24, then at step 116 the light sensing device 32 is reset via an internal clock to countdown until the next measurement cycle. However, returning to step 110, if the microprocessor 68 determines that a fourth invalid reading has occurred, and then at step 118, an alarm signal is sent to the depth control processor unit 24 indicates that there is an error in the readings being performed by the light sensing device 32, and maintenance or replacement may be required.

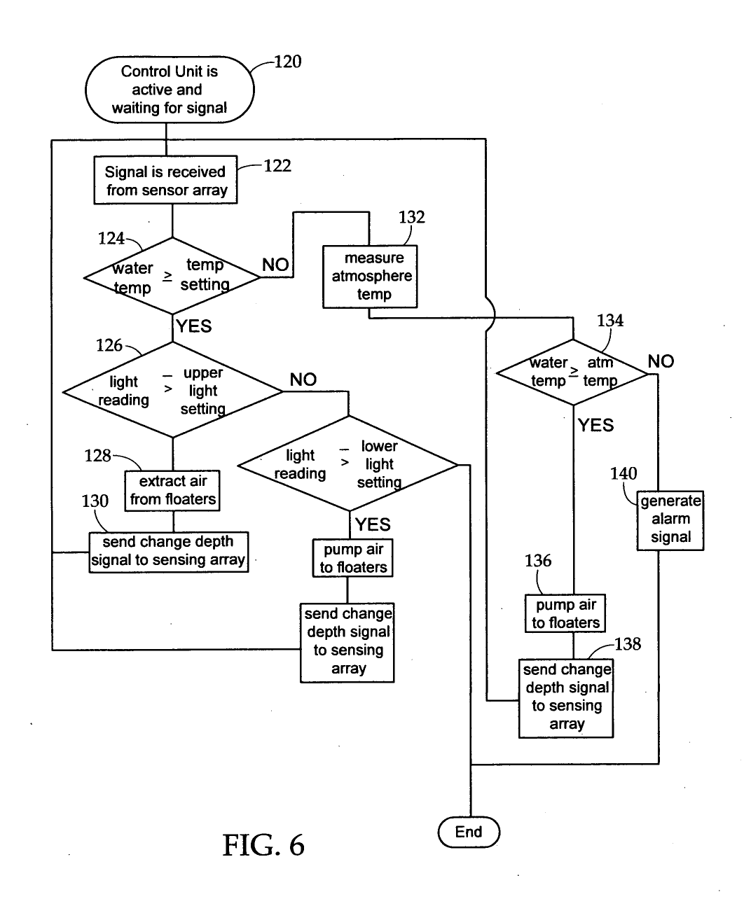

FIG. 6 illustrates the operational system flowchart for data communication between the depth control processor unit 24 and the solenoid valve 34 of the buoyancy support system 16 designed to control the increase or decrease the amount of air contained within the floating supports 22 based on the real-time light and water temperature readings taken by the light sensing device 32. The purpose of the data communication between the depth control processor unit 24 and the solenoid valve 34 is to allow for depth modification of the array of vessels 14 depending on the amount of light irradiation received by the array of vessels 14 to ensure optimal growth conditions.

At step 120, the depth control processor unit 24 is operable and is waiting to receive a signal from the light sensing device 32 regarding the current light and temperature conditions in which the system 10 is deployed. At step 122, the depth control processor unit 24 receives the signal sent from the light sensing device through the antenna 90C of the depth control processor unit 32.

The first analysis of the readings occurs at step 124 to determine if the current water temperature is greater than or equal to the original temperature setting for optimal growth within the vessels 14. If the current water temperature is greater than or equal to the original temperature setting, then at step 126 the next determination is made concerning whether the light irradiation levels are greater than or equal to the original light setting. When the light reading is greater than or equal to the light setting, coupled with the temperature reading determined above to be greater than or equal to the temperature setting, the system at step 128 extracts air from the floating supports 22 (see discussion below regarding method for air extraction). Subsequently, once air has been removed from the floating supports 22, at step 130, the depth control processor unit 24 sends a change depth signal to the light sensing device 32 to constantly monitor the light and temperature readings until the original preset conditions have been achieved. Once the conditions have been met, the system returns to the main loop at step 120 waiting for the next reading from the light sensing device 32.

Returning to step 124, if the water temperature is less than the original temperature setting, then at step 132 a measurement of the atmospheric temperature will be taken. Once the atmospheric temperature has been measured, at step 134 the depth control processor unit 24 determines whether the current water temperature is less than the current atmospheric temperature. If the current water temperature is less than the atmospheric temperature, the depth control processor 24 will send a signal (see discussion below) at step 136 to pump air into the floating supports 22. Subsequently, once air has been pumped into the floating supports 22, at step 138, the depth control processor unit 24 sends a change depth signal to the light sensing device 32 to constantly monitor the light and temperature readings until the original preset conditions have been achieved. Once the conditions have been met, the system returns to the main loop at step 120 waiting for the next reading from the light sensing device 32. Alternatively, if the current water temperature is not less than the atmospheric temperature, then at step 140 an alarm signal will be sent to the depth control processor unit 24 indicating either a problem with the overall system 10 or unfavorable weather conditions (i.e. a hurricane) wherein the system 10 enters safety mode (see discussion above) for overall security. Lastly, returning to step 126, after it has been determined the current light reading is less than the upper light setting, at step 140, the depth control processor unit 24 determines whether the current light reading is less than the lowest light setting permitted. If the light reading is below

FIG. 7 illustrates the harvesting and feeding operations of the instant invention, specifically how biomass is extracted from the vessels 14 along with treating the micro algae with various nutrients and metabolic catalysts. In the preferred embodiment, the feed/harvest control unit 46 includes a three hundred fifteen or four hundred eighteen MHz short range transmitter and a receiver to communicate with the depth control processor unit 24.

As described above, the feed/harvest control unit 46 is in data and electrical communication with the depth control processor 24 for a variety of functions, including, but not limited to: (1) It might receive wireless orders to change the harvesting periods in response to a command sent to the depth control processor 24 from a remote location or to react to different environmental conditions measured by the depth control (for example, if there is very low light period, like in a rainy day, then the period between harvests might be increased to give enough time to the micro-algae to reproduce); (2) It might receive wireless orders to initiate an emergency harvesting routine in order to minimize potential environmental damages in case of a storm or hurricane (for example the depth control processor 24 will sink the array 14, but the array 14 would not have a high density of micro-algae due to the emergency harvesting routine triggered by the feed/harvest control unit 46, so there would not be a significant amount of biomass release to the environment in case the array is damaged by the storm); (3) The feed/harvest control unit 46 will send a wireless signal to the depth control processor 24 in case there is a problem with the harvesting or feeding process, so the maintenance crews could be alerted to take care of the problem.

In the preferred embodiment, the feed/harvest control unit 46 starts the harvesting and feeding operations of the system 10 at a preset time, given the overall operating parameters of the system 10. In an alternate embodiment, an electronic timer is substituted for the feed/harvest control unit 46 to determine when the operation begins. Initially, the control unit 46 activates the solenoid valve 48 located on the harvesting system 18, thereby opening the air valve 50 to allow treated water to flow through, while simultaneously activating the motor 54 of the pump 52 to begin pumping water into the array of vessels 14. The treated water, which contains nutrients, carbon dioxide and metabolic catalysts (see discussion below) reach the vessels 14 via the nutrient piping 44 for biomass production. While treated water enters the vessels 14, water with micro algae biomass already contained within the vessels 14 is pushed toward a filter 142 that captures and stores the biomass for a predetermined amount of time, preferably twenty-four hours. The drained water from the filter 142 is stored at the bottom of the biomass storage tank 58, and subsequently is suctioned by the pump 52 of the feeding and harvesting system 18. The pressured water passes through a venture device 144, which preferably functions as a siphon in order to add new water to the system 10 from the fluid medium 20. Water is then treated with ozone in order to minimize any parasitic micro algae that may have entered the system from the external fluid entering the system. Lastly, nutrients, carbon dioxide and metabolic catalysts are introduced into the water prior to introduction into the vessels 14, depending on when the solenoid 48 activates the air valve 50 again.

FIG. 8 illustrates the connection between the array of vessels 14 and the nutrient piping 44, and the harvesting piping 60. As described above, nutrients, carbon dioxide and other catalysts are delivered to the vessels 14 via the nutrient piping 44, wherein the piping 44 extends the length of one of the openings of the array 14. In the preferred embodiment, the nutrient piping 44 includes a plurality of holes for the nutrients to pass from the piping 44 to the micro algae located within the vessels 14. At the other end of the array, the harvesting piping 60 extends to capture the biomass and transport it to the storage tank 60. As described in FIG. 7, the harvesting/feeding system 18 functions as a continuous cycle feeding nutrients in one end and suctioning biomass out from the other end of the array while constantly introducing treated water into the array 14.

FIG. 9 illustrates an alternate embodiment of the instant invention, wherein the system 10 includes a support structure 12 (see FIG. 2), wherein the support structure 12 is able to accommodate an array of vessels 14 capable of receiving light. A plurality of buoyancy support systems 146 are in communication with the array of vessels 14, wherein each support system 146 regulates the amount of light introduced into the vessels 14 within the fluid medium 20.

Each buoyancy support system 146 further includes a floating support 148, wherein the floating support 148 preferably resides on the surface of the fluid medium 20 to provide stability to the system 10 and allow for the array of vessels 14 to remain substantially constant in position. Furthermore each floating support 148 includes a depth control riser 150 that works in conjunction with a buoyancy depth controller 152 via a connecting line 154 to regulate the depth of the array of vessels 14 within the fluid medium 20. Additionally, each floating support 148 includes an air compressor 156, wherein the compressor 156 allows for the introduction of compressed air into each depth controller 152 via a compressed air piping 158 such that altering the amount of compressed air within the depth controller 152 causes the array of vessels 14 to receive varying amounts of light. The buoyancy support system 146 further comprises a depth control processing unit 160 attached to the floating support 148, wherein the depth control processing unit 160 regulates the amount of compressed air introduced into each depth controller 152 based on a signal sent from a plurality of light-sensing devices 32 (see FIG. 3) located within the array of vessels 14. Each light sensing device 32 measures the amount of light received by the array 14 and subsequently transmits the signal to the depth control processing unit 160 such that the processing unit 160 determines whether the amount of light received by the array 14 is within normal operating parameters, or alternatively whether the distance between the vessels 14 and the surface of the fluid medium 20 must be adjusted. As stated above, the depth of the vessels 14 is adjusted by the system 10 introducing compressed air into the buoyancy depth controllers 152 via the piping 158 and allowing the vessels to rise or fall by the depth control risers 150.

### Experimental Data

Based on the notion obtained from the previous design of experiments that shows the importance of light irradiation in the biomass yields for the system it is necessary to obtain the values for the upper and lower light settings for a micro-algae array. In order to do that, a strain of cultured micro-algae (in this case chlorella vulgaris that is fairly common in most oceans) was inoculated in 5 1 liter vessels that contain seawater previously treated with ozone in order to reduce the likelihood of parasitic algal growth. A sample of the same pre-treated seawater was placed in a 5 separate 1 liter vessels to serve as blank reference samples to prove the effectiveness of the seawater pre-treatment. Each inoculated vessel was paired with a reference blank vessel and was fed with the same amount of nutrients and placed under regulated light with difference luminance settings. The first group was placed under an equivalent light of 10 W/m2 (2381 lux), the second group was placed under 20 W/m2 and so on until the fifth group was under a 50 W/m2 light. A 100 ml sample to determine the density of biomass in every vessel in grams/liter was taken every 12 hours for 5 days, every sample was taken with a previously sterilized instrument and was returned to its respective vessel to decrease the influence of extraction rate in biomass growth. This experiment showed that light below 20 W/m2 (4762 lux) produced poor results in biomass development (at 10 W/m2 there was half the density of biomass than at 20 W/m2), however algae density results at 30, 40 and 50 W/m2 were not significantly different than those observed at 20 W/m2. This indicates that the inflection point for the quadratic component of light irradiation occurs between 20 and 30 W/m2 for this species. No observable algae growth occurred in any of the reference blank vessels that were not inoculated with the micro-algae strain.

A second battery of experiments was conducted with 10 groups with 5 one liter-vessels per group with inoculated strains of micro algae. The same feeding and sampling procedure was applied to each one of the vessels in any group, but this tie 5 groups were exposed to low light conditions in order to determine when the photosynthetic light saturation effect starts to occur and 5 were exposed to high light irradiance in order to determine the sun radiation tolerance of this micro-algae strain. Each group composed of 5 vessels (to compensate for vessel to vessel variation) was exposed to different irradiation levels: 10 W/m2, 15 W/2, 20 W/m2, 25 W/m2, 30 W/m2, 50 W/m2, 75 W/m2, 100 W/m2, 125 W/m2 and 150 W/m2.

The results showed a significant growth between the 15 W/m2 and the 20 W/m2 compared to the 10 W/m2 and a slight but perceptible biomass growth of the 25 W/m2 over the 20 W/m2. However the results for the 25 W/2 were almost the same as the 30 W/m2, which indicates that for this species light saturation starts to occur definitely at 25 W/m2. The small difference between the 15 and 20 W/m2 indicates that the lowest allowable setting for light is between these 2 values, so it could be set at approximately 18 W/m2. In the highly irradiated vessels, there was no significant difference between the 50 and 75 W/m2 vessels, a slight decrease in biomass density was observed at 100 W/m2 and a significant decrease in biomass density was observed at 125 and 150 W/m2. In this way, the upper limit for light setting could be set at 75 W/m2 and the minimum setting for light irradiation at 18 W/m2. Any value in between presents approximately similar biomass densities.

What is claimed:

1. A system for controlled exposure to light irradiation comprising:
a support structure;
an array of vessels disposed in a horizontal manner and substantially located upon the support structure;
a buoyancy support system in communication with the array of vessels, wherein the buoyancy support system regulates the amount of light introduced upon the vessels within a fluid medium; and
a feeding/harvesting system in communication with the array of vessels, wherein the feeding harvesting system regulates the growth of elements contained within the vessels.

2. The system of claim 1, wherein the buoyancy support system further comprises:
a floating support, wherein the floating support resides on the surface of the fluid medium;
a depth control processor unit, wherein the depth control processor unit is substantially located on the floating support;
a plurality of depth controllers, wherein the depth controllers are in fluid communication with the depth control processor unit via compressed air piping, to regulate the depth of the array of vessels within the fluid medium; and
a compressed air storage tank, wherein the tank allows for the introduction of compressed air into the depth controllers via the air piping.

3. The system of claim 1, wherein the feeding harvesting system further comprises:
a floating support, wherein the floating support resides on the surface of the fluid medium;
a nutrient storage tank, wherein the nutrient storage tank allows for the introduction of various nutrients into the vessels;
a plurality of nutrient piping, in fluid communication with the nutrient storage tank and the array of vessels for transporting nutrients into the vessels;
a biomass storage tank, wherein the biomass storage tank allows for the collection of biomass produced by the light irradiation system;
a plurality of harvesting piping, in fluid communication with the biomass storage tank and the array of vessels for transporting of biomass into the tank; and
a feed harvest control unit, wherein the feed harvest control unit regulates the introduction of nutrients into the vessels and the removal of biomass from the system.

4. The system of claim 1, wherein a plurality of light-sensing devices are located within the array of vessels such that each light-sensing device measures the amount of light received by the array of vessels.

5. The system of claim 2, wherein the floating support of the buoyancy support system further comprises:
a solenoid operated valve, wherein the solenoid operated valve regulates an air valve to maintain air flow in and out of the depth controllers; and
a pump disposed to transfer air from the compressed air storage tank.

6. The system of claim 3, wherein the floating support of the feeding harvesting system further comprises:
a solenoid operated valve, wherein the solenoid operated valve controls an air valve; and
a pump, wherein the pump activates a motor to regulate the introduction of nutrients into the vessels, and is in fluid communication with the air valve.

7. The system of claim 1, wherein a quantity of micro algae are placed within the vessels for the production of biomass.

8. The system of claim 1, wherein the fluid medium is a body of water.

9. The system of claim 5, wherein the depth control processor unit is in electrical and data communication with at least one light-sensing device and is disposed to regulate the depth of the array of vessels by the introduction of compressed air into the vessels.

10. The system of claim 5, wherein the depth control processor unit is in electrical communication with the pump and solenoid valve of the buoyancy support system and is disposed to activate the air valve based on the light reading from the light-sensing device.

11. The light irradiation system of claim 6, wherein the feed harvest control unit is in data and electrical communication with the depth control processor unit and is disposed to regulate the harvesting and feeding cycle of the system.

12. The light irradiation system of claim 6, wherein the feed harvest control unit simultaneously activates the solenoid valve of the feeding harvesting system along with the motor of the pump and is disposed to begin pumping water into the vessels.

13. A system for controlled exposure to light irradiation comprising:
a support structure;
an array of vessels disposed in a horizontal manner and substantially located upon the support structure; and
a plurality of buoyancy support systems in communication with the array of vessels, wherein each buoyancy support system regulates the amount of light introduced upon the vessels within a fluid medium.

14. The system of claim 13, wherein each buoyancy support system further comprises:
a floating support, wherein the floating support further includes:
a depth control riser, wherein the depth control riser allows for movement of the array of vessels; and
an air compressor for introduction of compressed air into each vessel; and
a buoyancy depth controller, wherein the depth controller is in communication with the depth control riser and is disposed to regulate the distance between the array of vessels and each floating support.

15. The system of claim 14, wherein micro algae is introduced into the vessels for biomass production.

16. The system of claim 15, wherein the fluid medium is the ocean.
